(19) United States
(12) Patent Application Publication
(10) Pub. No.: US 2005/0096324 A1
(43) Pub. Date: May 5, 2005

(54) Macrocyclic kinase inhibitors

(76) Inventors: Zhi-Fu Tao, Gurnee, IL (US); Nan-Horng Lin, Vernon Hills, IL (US); Le Wang, Vernon Hills, IL (US); Thomas J. Sowin, Wadsworth, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/702,140

(22) Filed: Nov. 5, 2003

(51) Int. Cl. A61K 31/498; C07D 491/14

(52) U.S. Cl. 514/250; 540/456; 540/460

(57) ABSTRACT

Compounds having the formula 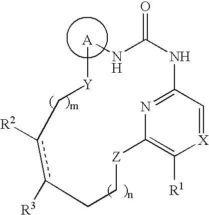 are useful for inhibiting protein kinases. Also disclosed are methods of making the compounds, compositions containing the compounds, and methods of treatment using the compounds.

### TECHNICAL FIELD

The present invention relates to substituted ureas which are useful for inhibiting protein kinases, methods of making the compounds, compositions containing the compounds, and methods of treatment using the compounds.

### BACKGROUND OF THE INVENTION

Protein kinases have been clearly shown to be important in the progression of many disease states that are induced by the inappropriate proliferation of cells. These kinases are often found to be up-regulated in many hyperproliferative states such as cancer. These kinases may be important in cell signaling, where their inappropriate activation induces cells to proliferate (e.g., EGFR, ERBB2, VEGFR, FGFR, PDGFR, c-Met, IGF-1R, RET, TIE2). Alternatively, they may be involved in signal transduction within cells (e.g., c-Src, PKC, Akt, PKA, c-Abl, PDK-1). Often these signal transduction genes are recognized proto-oncogenes. Many of these kinases control cell cycle progression near the G1-S transition (e.g., Cdk2, Cdk4), at the G2-M transition (e.g., Wee1, Myt1, Chk1, Cdc2) or at the spindle checkpoint (Plk, Aurora1 or 2, Bub1 or 3). Furthermore, kinases are intimately linked to the DNA damage response (e.g., ATM, ATR, Chk1, Chk2). Deregulation of these cellular functions: cell signaling, signal transduction, cell cycle control, and DNA repair, are all hallmarks of hyperproliferative diseases, particularly cancer. It is therefore likely that pharmacological modulation of one or more kinases would be useful in slowing or stopping disease progression in these diseases.

### SUMMARY OF THE INVENTION

In its principle embodiment the present invention provides a compound of formula (I) 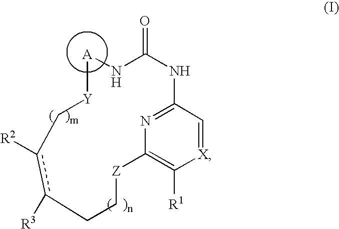
or a therapeutically acceptable salt thereof, wherein 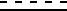
is a single or double bond;

A is selected from the group consisting of aryl and heteroaryl, wherein the aryl and the heteroaryl are optionally substituted with one, two, or three substituents independently selected from the group consisting of alkoxy, alkyl, cyano, halo, hydroxy, and NR^{a}R^{b};

R¹ is selected from the group consisting of hydrogen, alkoxycarbonyl, alkyl, carboxy, cyano, halo, and nitro;

R² and R³ are independently selected from the group consisting of hydrogen, alkoxy, alkoxycarbonyl, alkyl, alkylsulfonyl, arylsulfonyl, halo, hydroxy, and NR^{a}R^{b};

X is selected from the group consisting of CH and N;

Y and Z are independently selected from the group consisting of CH₂, O, S, and NR^{z}, wherein R^{z} is selected from the group consisting of hydrogen and alkyl; and

the sum of m and n is between 0 and 6, inclusive.

In another embodiment the present invention provides a compound of formula (II) 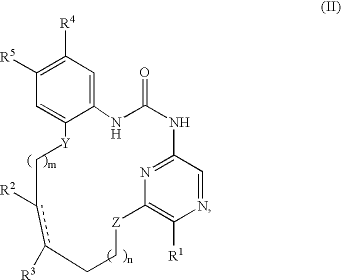
or a therapeutically acceptable salt thereof, wherein 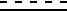
is a single or double bond;

R¹ is selected from the group consisting of hydrogen, alkoxycarbonyl, alkyl, carboxy, cyano, halo, and nitro;

R² and R³ are independently selected from the group consisting of hydrogen, alkoxy, alkoxycarbonyl, alkyl, alkylsulfonyl, arylsulfonyl, halo, hydroxy, and NR^{a}R^{b};

R⁴ is selected from the group consisting of alkoxy, alkyl, cyano, halo, hydroxy, and NR^{a}R^{b};

R⁵ is selected from the group consisting of hydrogen, alkoxy, alkoxyalkyl, alkoxycarbonyl, alkoxycarbonylalkyl, alkylsulfanyl, arylsulfanyl, alkylsulfonyl, arylsulfonyl, carboxy, carboxyalkyl, hydroxy, hydroxyalkyl, NR^{a}R^{b}, (NR^{a}R^{b})alkoxy, (NR^{a}R^{b})alkyl, (NR^{a}R^{b})carbonyl, (NR^{a}R^{b})carbonylalkoxy, and (NR^{a}R^{b})carbonylalkyl;

Y and Z is independently selected from the group consisting of CH₂, O, S, and NR^{z}, wherein R^{z} is selected from the group consisting of hydrogen and alkyl; and

the sum of m and n is between 0 and 6, inclusive.

In a preferred embodiment the present provides a compound of formula (II) wherein

R¹ is selected from the group consisting of hydrogen and cyano;

R² and R³ are independently selected from the group consisting of hydrogen and hydroxy;

R⁴ is halo;

R⁵ is hydrogen;

Y is O;

Z is O; and

mis 1.

In another preferred embodiment the present invention provides a compound of formula (I) wherein 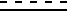
is a single or double bond;

A is selected from the group consisting of aryl and heteroaryl, wherein the aryl and the heteroaryl are optionally substituted with one, two, or three substituents independently selected from the group consisting of alkoxy, alkyl, cyano, halo, hydroxy, and NR^{a}R^{b};

R¹ is selected from the group consisting of hydrogen, alkyl, cyano, and halo;

R² and R³ are independently selected from the group consisting of hydrogen, alkoxy, alkoxycarbonyl, alkyl, alkylsulfonyl, arylsulfonyl, halo, hydroxy, and NR^{a}R^{b};

X is selected from the group consisting of CH and N; and

mis 1 and n is 0.

Compounds which support this embodiment are
7-chloro-11,16-dioxa-2,4,19,21-tetraazatricyclo[15.3.1.0^{5,10}]henicosa-1(21),5,7,9,13,17,19-heptaen-3-one;
7-chloro-11,16-dioxa-2,4,19,21-tetraazatricyclo[15.3.1.0^{5,10}]henicosa-1(21),5,7,9,17,19-hexaen-3-one; and
7-chloro-13,14-dihydroxy-11,16-dioxa-2,4,19,21-tetraazatricyclo[15.3.1.0^{5,10}]henicosa-1(21),5,7,9,17,19-hexaen-3-one.

In another preferred embodiment the present invention provides a compound of formula (I) wherein 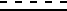
is a single or double bond;

A is selected from the group consisting of aryl and heteroaryl, wherein the aryl and the heteroaryl are optionally substituted with one, two, or three substituents independently selected from the group consisting of alkoxy, alkyl, cyano, halo, hydroxy, and NR^{a}R^{b};

R¹ is selected from the group consisting of hydrogen, alkyl, cyano, and halo;

R² and R³ are independently selected from the group consisting of hydrogen, alkoxy, alkoxycarbonyl, alkyl, alkylsulfonyl, arylsulfonyl, halo, hydroxy, and NR^{a}R^{b};

X is selected from the group consisting of CH and N; and

m is 1 and n is 1.

Compounds which support this embodiment are
7-chloro-11,17-dioxa-2,4,20,22-tetraazatricyclo[16.3.1.0^{5,10}]docosa-1(22),5,7,9,13,18,20-heptaen-3-one;
7-chloro-11,17-dioxa-2,4,20,22-tetraazatricyclo[16.3.1.0^{5,10}]docosa-1(22),5,7,9,18,20-hexaen-3-one;
7-chloro-13,14-dihydroxy-11,17-dioxa-2,4,20,22-tetraazatricyclo[16.3.1.0^{5,10}]docosa-1(22),5,7,9,18,20-hexaen-3-one;
7-chloro-3-oxo-11,17-dioxa-2,4,20,22-tetraazatricyclo[16.3.1.0^{5,10}]docosa-1(22),5,7,9,13,18,20-heptaene-19-carbonitrile;
7-chloro-3-oxo-11,17-dioxa-2,4,20,22-tetraazatricyclo[16.3.1.0^{5,10}]docosa-1(22),5,7,9,18,20-hexaene-19-carbonitrile; and
7-chloro-13,14-dihydroxy-3-oxo-11,17-dioxa-2,4,20,22-tetraazatricyclo[16.3.1.0^{5,10}]docosa-1(22),5,7,9,18,20-hexaene-19-carbonitrile.

In another embodiment the present invention provides a pharmaceutical composition comprising a compound of claim 1 or a therapeutically acceptable salt thereof, in combination with a therapeutically acceptable carrier.

In another embodiment the present invention provides a method for inhibiting protein kinases in a patient in recognized need of such treatment comprising administering to the patient a therapeutically acceptable amount of a compound of claim 1, or a therapeutically acceptable salt thereof.

In another embodiment the present invention provides a method for treating cancer in a patient in recognized need of such treatment comprising administering to the patient a therapeutically acceptable amount of a compound of claim 1, or a therapeutically acceptable salt thereof.

### DETAILED DESCRIPTION OF THE INVENTION

All publications, issued patents, and patent applications cited herein are hereby incorporated by reference.

As used in the present specification the following terms have the meanings indicated:

As used herein, the singular forms “a”, “an”, and “the” include plural reference unless the context clearly dictates otherwise.

The term “alkoxy,” as used herein, refers to an alkyl group attached to the parent molecular moiety through an oxygen atom.

The term “alkoxyalkyl,” as used herein, refers to an alkyl group substituted with at least one alkoxy group.

The term “alkoxycarbonyl,” as used herein, refers to an alkoxy group attached to the parent molecular moiety through a carbonyl group.

The term “alkoxycarbonylalkyl,” as used herein, refers to an alkyl group substituted with at least one alkoxycarbonyl group.

The term “alkyl,” as used herein, refers to a group derived from a straight or branched chain saturated hydrocarbon containing from one to ten carbon atoms. Preferred alkyl groups contain from one to four carbon atoms.

The term “alkylcarbonyl,” as used herein, refers to an alkyl group attached to the parent molecular moiety through a carbonyl group.

The term “alkylsulfanyl,” as used herein, refers to an alkyl group attached to the parent molecular moiety through a sulfur atom.

The term “alkylsulfonyl,” as used herein, refers to an alkyl group attached to the parent molecular moiety through a sulfonyl group.

The term “aryl,” as used herein, refers to a phenyl group, or a bicyclic or tricyclic fused ring system wherein one or more of the fused rings is a phenyl group. Bicyclic fused ring systems are exemplified by a phenyl group fused to a monocyclic cycloalkenyl group, as defined herein, a monocyclic cycloalkyl group, as defined herein, or another phenyl group. Tricyclic fused ring systems are exemplified by a bicyclic fused ring system fused to a monocyclic cycloalkenyl group, as defined herein, a monocyclic cycloalkyl group, as defined herein, or another phenyl group. Representative examples of aryl groups include, but are not limited to, anthracenyl, azulenyl, fluorenyl, indanyl, indenyl, naphthyl, phenyl, and tetrahydronaphthyl. The aryl groups of the present invention can be optionally substituted with one, two, three, four, or five substituents independently selected from the group consisting of alkoxy, alkyl, cyano, halo, haloalkoxy, haloalkyl, hydroxy, nitro, NR^{a}R^{b}, and oxo.

The term “arylalkyl,” as used herein, refers to an alkyl substituted with at least one aryl group.

The term “arylsulfanyl,” as used herein, refers to an aryl group attached to the parent molecular moiety through a sulfur atom.

The term “arylsulfonyl,” as used herein, refers to an aryl group attached to the parent molecular moiety through a sulfonyl group.

The term “carbonyl,” as used herein, refers to —C(O)—.

The term “carboxy,” as used herein, refers to —CO₂H.

The term “carboxyalkyl,” as used herein, refers to an alkyl group substituted with at least one carboxy group.

The term “cyano,” as used herein, refers to —CN.

The terms “halo” and “halogen,” as used herein, refer to F, Cl, Br, or I.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “heteroaryl,” as used herein, refers to an aromatic five- or six-membered ring where at least one atom is selected from the group consisting of N, O, and S, and the remaining atoms are carbon. The five-membered rings have two double bonds, and the six-membered rings have three double bonds. The heteroaryl groups are connected to the parent molecular moiety through a substitutable carbon or nitrogen atom in the ring. The term “heteroaryl” also includes bicyclic systems where a heteroaryl ring is fused to a phenyl group, a monocyclic cycloalkenyl group, as defined herein, a monocyclic cycloalkyl group, as defined herein, a monocyclic heterocyclyl group, as defined herein, or an additional monocyclic heteroaryl group; and tricyclic systems where a bicyclic system is fused to a phenyl group, a monocyclic cycloalkenyl group, as defined herein, a monocyclic cycloalkyl group, as defined herein, a heterocyclyl group, as defined herein, or an additional monocyclic heteroaryl group. Representative examples of heteroaryl groups include, but are not limited to, benzoxadiazolyl, benzoxazolyl, benzofuranyl, benzothienyl, cinnolinyl, dibenzofuranyl, furanyl, imidazolyl, indazolyl, indolyl, isoxazolyl, isoquinolinyl, isothiazolyl, naphthyridinyl, oxadiazolyl, oxadiazolyl, oxazolyl, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, triazinyl, and the like. The heteroaryl groups of the present invention can be optionally substituted with one, two, three, four, or five substituents independently selected from the group consisting of alkoxy, alkyl, cyano, halo, haloalkoxy, haloalkyl, hydroxy, nitro, NR^{a}R^{b}, and oxo.

The term “heteroarylalkyl,” as used herein, refers to a heteraryl group substituted with at least one heteroaryl group.

The term “hydroxy,” as used herein, refers to —OH.

The term “hydroxyalkyl,” as used herien, refers to an alkyl group substituted with at least one hydroxy group.

The term “nitro,” as used herein, refers to —NO₂.

The term “NR^{a}R^{b},” as used herein, refers to two groups, R^{a} and R^{b}, which are attached to the parent molecular moiety through a nitrogen atom. R^{a} and R^{b} are independently selected from the group consisting of hydrogen, alkoxycarbonyl, alkyl, alkylcarbonyl, aryl, arylalkyl, heteroaryl, heteroarylalkyl, and (NR^{c}R^{d})alkylcarbonyl, wherein the aryl, the aryl part of the arylalkyl, the heteroaryl, and the heteroaryl part of the heteroarylalkyl can be optionally substituted with one, two, three, four, or five substituents independently selected from the group consisting of alkoxy, alkyl, cyano, halo, haloalkoxy, haloalkyl, hydroxy, and nitro.

The term “(NR^{a}R^{b})alkoxy,” as used herein, refers t o an NR^{a}R^{b} up attached to the parent molecular moiety through an alkoxy group.

The term “(NR^{a}R^{b})alkyl,” as used herein, refers to an alkyl group substituted with at least one NR^{a}R^{b} group.

The term “(NR^{a}R^{b})carbonyl,” as used herein, refers to an NR^{a}R^{b} group attached to the parent molecular moiety through a carbonyl group.

The term “(NR^{a}R^{b})carbonylalkoxy,” as used herein, refers to an (NR^{a}R^{b})carbonyl group attached to the parent molecular moiety through an alkoxy group.

The term “(NR^{a}R^{b})carbonylalkyl,” as used herein, refers to an alkyl group substituted with at least one NR^{a}R^{b} group.

The term “NR^{c}R^{d},” as used herein, refers to two groups, R^{c} and R^{d}, which are attached to the parent molecular moiety through a nitrogen atom. R^{c} and R^{d} are independently selected from the group consisting of hydrogen and alkyl.

The term “(NR^{c}R^{d})alkyl,” as used herein, refers to an alkyl group substituted with at least one NR^{c}R^{d} group.

The term “(NR^{c}R^{d})alkylcarbonyl,” as used herein, refers to an (NR^{c}R^{d})alkyl group attached to the parent molecular moiety through a carbonyl group.

The term “oxo,” as used herein, refers to ═O.

The term sulfonyl, as used herein, refers to —SO₂—.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate,trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, or mixtures thereof, which possess the ability to inhibit protein kinases. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Because carbon-carbon double bonds exist in the present compounds, the invention contemplates various geometric isomers and mixtures thereof resulting from the arrangement of substituents around these carbon-carbon double bonds. It should be understood that the invention encompasses both isomeric forms, or mixtures thereof, which possess the ability to inhibit protein kinases. These substituents are designated as being in the E or Z configuration wherein the term “E” represents higher order substituents on opposite sides of the carbon-carbon double bond, and the term “Z” represents higher order substituents on the same side of the carbon-carbon double bond.

When it is possible that, for use in therapy, therapeutically effective amounts of a compound of formula (I), as well as therapeutically acceptable salts thereof, may be administered as the raw chemical, it is possible to present the active ingredient as a pharmaceutical composition. Accordingly, the invention further provides pharmaceutical compositions, which include therapeutically effective amounts of compounds of formula (I) or therapeutically acceptable salts thereof, and one or more pharmaceutically acceptable carriers, diluents, or excipients. The compounds of formula (I) and therapeutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of formula (I), or a therapeutically acceptable salt thereof, with one or more pharmaceutically acceptable carriers, diluents, or excipients.

Pharmaceutical formulations may be presented in unit dose forms containing a predetermined amount of active ingredient per unit dose. Such a unit may contain, for example, 0.5 mg to 1 g, preferably lmg to 700 mg, more preferably 5 mg to 100 mg of a compound of formula (I), depending on the condition being treated, the severity of the condition, the time of administration, the route of administration, the rate of excretion of the compound employed, the duration of treatment, and the age, gender, weight, and condition of the patient, or pharmaceutical formulations may be presented in unit dose forms containing a predetermined amount of an active ingredient per dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intramuscular, intravenous, or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, wasces, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an altenative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxy ethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of formula (I), and therapeutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

The compounds of formula (I) and therapeutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Phamaceutical Research, 3(6), 318 (1986).

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouth washes.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) for the treatment of neoplastic growth, for example colon or breast carcinoma, will generally be in the range of 0.1 to 100 mg/kg body weight of recipient (mammal) per day and more usually in the range of 1 to 10 mg/kg body weight per day.

Determination of Biological Activity

The Chkl enzymatic assay was carried out using recombinant Chk1 kinase domain protein covering amino acids from residue 1 to 289 and a polyhistidine tag at the C-terminal end. Human cdc25c peptide substrate contained a sequence from amino acid residue 204 to 225. The reaction mixture contained 25 mM of HEPES at pH 7.4, 10 mM MgCl₂, 0.08 mM Triton X-100, 0.5 mM DTT, 5 μM ATP, 4 nM 33P ATP, 5 μM cdc25c peptide substrate, and 6.3 nM of the recombinant Chk1 protein. Compound vehicle DMSO was maintained at 2% in the final reaction. After 30 minutes at room temperature, the reaction was stopped by addition of equal volume of 4M NaCl and 0.1M EDTA, pH 8. A 40 μL aliquot of the reaction was added to a well in a Flash Plate (NEN Life Science Products, Boston, Mass.) containing 160 μL of phosphate-buffered saline (PBS) without calcium chloride and magnesium chloride and incubated at room temperature for 10 minutes. The plate was then washed 3 times in PBS with 0.05% of Tween-20 and counted in a Packard TopCount counter (Packard BioScience Company, Meriden, Conn.).

Compounds of the present invention inhibited Chk1 at IC₅₀ values between about 5 nM and about 5 μM. Preferred compounds inhibited Chk1 at IC₅₀ values between about 5 nM and about 25 nM. Thus, the compounds of the invention are useful in treating disorders which are caused or exacerbated by increased protein kinase levels.

The compounds of the invention, including not limited to those specified in the examples, possess the ability to inhibit protein kinases. As protein kinase inhibitors, such compounds are useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compounds may also be useful in treating solid tumors arising from hematopoietic malignancies such as leukemias (i.e., chloromas, plasmacytomas and the plaques and tumors of mycosis fungicides and cutaneous T-cell lymphoma/leukemia) as well as in the treatment of lymphomas (both Hodgkin's and non-Hodgkin's lymphomas). In addition, these compounds may be useful in the prevention of metastases from the tumors described above either when used alone or in combination with radiotherapy and/or other chemotherapeutic agents.

Synthetic Methods

Abbreviations which have been used in the descriptions of the scheme and the examples that follow are: THF for tetrahydrofuran; PPh₃ for triphenylphosphine; and DMSO for dimethylsulfoxide.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. The groups A, R¹, R², R³, X, Y, Z, m, and n are as defined above unless otherwise noted below.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

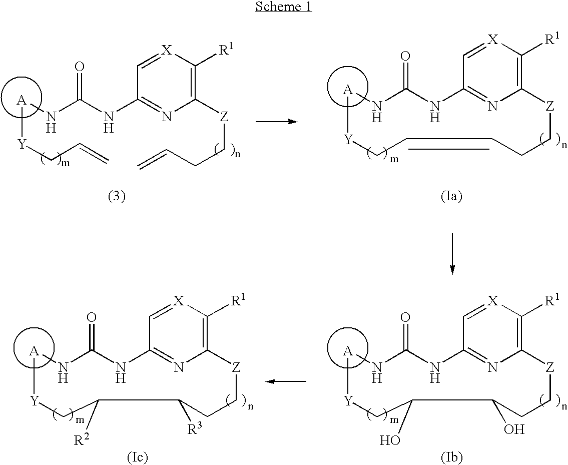

Scheme 1 shows the synthesis of compounds of formula (Ia), (Ib), and (Ic). Compounds of formula (3), which can be prepared by numerous methods known to those of ordinary skill in the art, can be converted to compounds of formula (Ia) by treatment with Grubbs' catalyst (first or second generation). Solvents typically used in this reaction include dichloromethane, chloroform, and methyl tert-butyl ether. The reaction is typically run at a temperature of about 50° C. to about 70° C. for about 12 to about 24 hours.

Compounds of formula (Ia) can be converted to compounds of formula (Ib) by treatment with an oxidizing agent such as OSO₄, KMnO₄, H₂O₂, N-methylmorpholine-N-oxide, and mixtures thereof. Examples of solvents used in these reactions include THF, water, 2-methyl-2-propanol, methyl tert-butyl ether, and mixtures thereof. The reaction is typically conducted at about 0° C. to about 30° C. for about 12 to about 24 hours.

The hydroxyl groups in the compounds of formula (Ib) can be converted to various other functional groups using methods known to those of ordinary skill in the art.

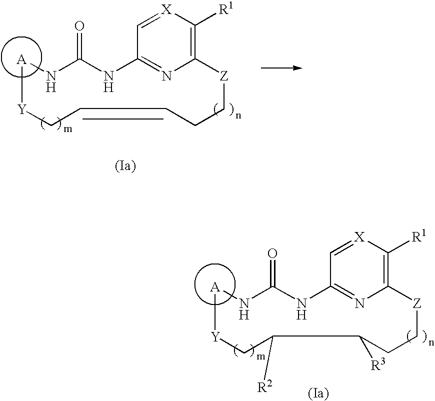

As shown in Scheme 2, compounds of formula (Ia) can be converted to compounds of formula (Id) where R² and R³ are hydrogen by hydrogenation using a metal catalyst in the presence of hydrogen. Representative palladium catalysts include Pd/C, RhCl(PPh₃)₃, and PtO₂. Examples of solvents used in these reactions include methanol, THF, ethanol, methyl tert-butyl ether, and mixtures thereof. The reaction is typically conducted at about 20° C. to about 40° C. for about 5 minutes to about 2 hours.

Compounds of formula (Ia) can be converted to compounds of formula (Id) where R² and R³ are selected from the group consisting of hydroxy and NR^{a}R^{b} by methods known to those of ordinary skill in the art. Compounds of formula (Id) where at least one of R² and R³ is either hydroxy or NR^{a}R^{b} can be further functionalized using methods known to those of ordinary skill in the art.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

Compounds of the invention were named by ACD/ChemSketch version 5.0 (developed by Advanced Chemistry Development, Inc., Toronto, ON, Canada) or were given names consistent with ACD nomenclature.

### EXAMPLE 1

### 7-chloro-11,17-dioxa-2,4,20,22-tetraazatricyclo[16.3.1.0^{5,10}]docosa-1(22),5,7,9,13,18,20-heptaen-3-one

### EXAMPLE 1A

### 2-(allyloxy)-5-chloroaniline

A mixture of 2-amino-4-chlorophenol (10 g, 69.65 mmol) and K₂CO₃ (14.53 g, 105 mmol) in acetone (160 mL) was treated with allyl bromide (9.03 mL, 104 mmol), stirred at room temperature overnight, and filtered. The filter cake was washed with acetone and the combined filtrates were concentrated. The residue was purified by flash column chromatography eluting with hexanes/ethyl acetate (10:1) to provide 8.31 g (65%) of the desired product. MS (DCI/NH₃) m/z 184.02 (M+H)⁺; ¹H NMR (500 MHz, DMSO-d₆) δ 4.51 (m, 2H), 5.02 (s, 2H), 5.24 (dd, J=10.53, 1.68 Hz, 1H), 5.42 (m, 1H), 6.04 (m, 1H), 6.47 (dd, J=8.54, 2.44 Hz, 1H), 6.66 (d, J=2.75 Hz, 1H), 6.75 (d, J=8.54 Hz, 1H).

### EXAMPLE 1B

### 1-(allyloxy)-4-chloro-2-isocyanatobenzene

A solution of 20% phosgene (5 mL, 47.3 mmol) in toluene (6 mL) at reflux was treated slowly with a solution of Example 1A (1 g, 5.44 mmol) in toluene (10 mL). The mixture was heated to reflux at 110° C. for 20 hours, cooled to room temperature, and concentrated to provide the desired product. ¹H NMR (500 MHz, CD₂Cl₂) δ 4.62 (d, J=5.30 Hz, 2H), 5.33 (dd, J=10.76, 1.40 Hz, 1H), 5.46 (dd, J=17.31, 1.40 Hz, 1H), 6.07 (m, 1H), 6.84 (d, J=8.73 Hz, 1H), 6.99 (d, J=2.50 Hz, 1H), 7.10 (dd, J=8.74, 2.50 Hz, 1H).

### EXAMPLE 1C

### 6-(3-butenyloxy)-2-pyrazinamine

A suspension of NaH (60%, 618 mg, 15.45 mmol) in dioxane (30 mL) at 0° C. was treated with 3-buten-1-ol (1.33 mL, 15.45 mmol), stirred for 2 hours, treated with 2-amino-6-chloropyrazine (1 g, 7.72 mmol), stirred at 100° C. for 2.5 days, cooled to room temperature, and diluted with ethyl acetate. The mixture was washed with water, dried (MgSO₄), filtered, and concentrated. The concentrate was purified by flash column chromatography eluting with hexanes/ethyl acetate (2:1) to provide the desired product (390 mg, 31 %). MS (DCI/NH₃) m/z 166.12 (M+H)⁺; ¹H NMR (500 MHz, benzene-d₆) δ 2.66 (m, 2H), 4.42 (t, J=6.87 Hz, 2H), 5.24 (dd, J=10.22, 1.98 Hz, 1H), 5.30 (m, 1H), 6.04 (m, 1H), 7.64 (s, 1H), 7.65 (s, 1H).

### EXAMPLE 1D

### N-[2-(allyloxy)-5-chlorophenyl]-N′-[6-(3-butenyloxy)-2-pyrazinyl]urea

A mixture of Example 1B (201 mg, 0.96 mmol) and Example 1C (158 mg, 0.96 mmol) in toluene (15 mL) was stirred at 110° C. for 15 hours and concentrated. The concentrate was purified by flash column chromatography eluting with hexanes/ethyl acetate (1:1) to provide the desired product (185 mg, 52%). MS (DCI/NH₃) m/z 375.12 (M+H)⁺; ¹H NMR (500 MHz, DMSO-d₆) δ 2.52 (m, 2H), 4.33 (t, J=6.55 Hz, 2H), 4.70 (d, J=5.30 Hz, 2H), 5.09 (dd, J=10.29, 1.25 Hz, 1H), 5.16 (dd, J=17.31, 1.40 Hz, 1H), 5.31 (d, J=10.61 Hz, 1H), 5.43 (dd, J=17.16, 1.25 Hz, 1H), 5.88 (m, 1H), 6.08 (m, 1H), 7.02 (d, J=8.75 Hz, 1H), 7.06 (dd, J=8.75 Hz, 2.5 Hz, 1H), 7.89 (s, 1H), 8.22 (d, J=2.50 Hz, 1H), 8.69 (s, 1H), 9.07 (s, 1H), 10.08 (s, 1H).

### EXAMPLE 1E

### 7-chloro-11,17-dioxa-2,4,20,22-tetraazatricyclo[16.3.1.0^{5,10}]docosa-1(22),5,7,9,13,18,20-heptaen-3-one

A mixture of Example 1D (60 mg, 0.16 mmol) in CH₂Cl₂ (66 mL) was treated with the second generation Grubbs' catalyst (20 mg, 0.024 mmol), stirred at 50° C. overnight, and concentrated. The residue was purified by flash column chromatography eluting with hexanes/ethyl acetate (1:1) to provide the desired product (22 mg, 40%). MS (DCI/NH₃) m/z 347.11 (M+H)⁺; ¹H NMR (500 MHz, CD₂Cl₂) δ 2.70 (d, J=7.25 Hz, 2H), 4.63 (m, 4H), 6.05 (m, 2H), 6.90 (d, J=8.73 Hz, 1H), 7.02 (dd, J=8.73, 2.57 Hz, 1H), 7.15 (s, 1H), 7.72 (s, 1H), 7.84 (s, 1H), 8.25 (d, J=2.57 Hz, 1H), 10.55 (s, 1H).

### EXAMPLE 2

### 7-chloro-11,17-dioxa-2,4,20,22-tetraazatricyclo[16.3.1.0^{5,10}]docosa-1(22),5,7,9,18,20-hexaen-3-one

A suspension of Pt/C (10%, 2 mg) in 3:1 methanol/THF (3 mL) was treated with Example 1E (17 mg, 0.049 mmol). The reaction mixture was bubbled with hydrogen for 10 minutes and filtered through diatomaceous earth (Celite ). The filtrate was concentrated and the residue was purified by recrystallization from ethyl acetate to provide the desired product (13.7 mg, 80%). MS (DCI/NH₃) m/z 349.11 (M+H)⁺; ¹H NMR (500 MHz, DMSO-d₆) δ 1.64 (m, 2H), 1.84 (m, 2H), 1.89 (m, 2H), 4.14 (t, J=5.16 Hz, 2H), 4.48 (t, J=7.80 Hz, 2H), 7.08 (d, J=2.50 Hz, 1H), 7.09 (s, 1H), 7.89 (s, 1H), 7.95 (s, 1H), 8.23 (d, J=2.50 Hz, 1H), 10.26 (s, 1H), 10.32 (s, 1H).

### EXAMPLE 3

### 7-chloro-13,14-dihydroxy-11,17-dioxa-2,4,20,22-tetraazatricyclo[16.3.1.0^{5,10}]docosa-1(22),5,7,9,18,20-hexaen-3-one

A solution of Example 1E (20 mg, 0.081 mmol) and N-methylmorpholine-N-oxide (14 mg, 0.12 mmol) in THF (1.6 mL) and H₂O (0.2 mL) at 0° C. was treated with 2.5 wt % of OsO₄ in 2-methyl-2-propanol (0.065 mL), stirred overnight at room temperature, and filtered. The filter cake was washed with water and dried to provide the desired product (22 mg, 73%). MS (DCI/NH₃) m/z 381.1 (M+H)⁺; ¹H NMR (500 MHz, DMSO-d₆) δ 1.84 (m, 1H), 2.29 (m, 1H), 3.82 (m, 2H), 4.12 (m, 2H), 4.46 (m, 2H), 4.57 (m, 2H), 7.11 (dd, J=8.73 Hz, 2.50 Hz, 1H), 7.15 (d, J=9.05 Hz, 1H), 7.85 (s, 1H), 7.93 (s, 1H), 8.17 (d, J=2.81 Hz, 1H), 10.03 (s, 1H), 10.25 (s, 1H).

### EXAMPLE 4

### 7-chloro-11,16-dioxa-2,4,19,21-tetraazatricyclo[15.3.1.0^{5,10}]henicosa-1(21),5,7,9,13,17,19-heptaen-3-one

### EXAMPLE 4A

### 6-(allyloxy)-2-pyrazinamine

A mixture of allyl alcohol (0.21 mL, 3.08 mmol) in dioxane (4 mL) was treated with NaH (60%, 3.08 mmol), stirred for 30 minutes, treated with 2-amino-6-chloropyrazine (200 mg, 1.54 mmol), heated to 140° C. in a Smith Synthesizer for 2200 seconds, and filtered. The filtrate was concentrated and purified by flash column chromatography eluting with hexanes/ethyl acetate (2:1) to provide the desired product (133 mg, 57%). MS (DCI/NH₃) m/z 152.0 (M+H)⁺; ¹H NMR (500 MHz, CD₂Cl₂) δ 4.75 (m, 2H), 5.24 (m, 1H), 5.37 (m, 1H), 6.05 (m, 1H), 7.50 (s, 1H), 7.53 (s, 1H).

### EXAMPLE 4B

### N-[2-(allyloxy)-5-chlorophenyl]-N′-[6-(allyloxy)-2-pyrazinyl]urea

The desired product was prepared (650 mg, 66% yield) by substituting Example 4A for Example 1C in Example 1D. MS (DCI/NH₃) m/z 361.1 (M+H)⁺; ¹H NMR (400 MHz, DMSO-d₆) δ 4.69 (d, J=5.52 Hz, 2H), 4.82 (d, J=5.52 Hz, 2H), 5.28 (m, 2H), 5.38 (dd, J=8.29, 1.53 Hz, 1H), 5.43 (dd, J=8.13, 1.69 Hz, 1H), 6.07 (m, 2H), 7.01 (m, 1H), 7.05 (m, 1H), 7.93 (s, 1H), 8.21 (d, J=2.45 Hz, 1H), 8.69 (s, 1H), 9.07 (s, 1H), 10.08 (s, 1H).

### EXAMPLE 4C

### 7-chloro-11,16-dioxa-2,4,19,21-tetraazatricyclo[15.3.1.0^{5,10}]henicosa-1(21),5,7,9,13,17,19-heptaen-3-one

The desired product was prepared (144 mg, 56% yield) by substituting Example 4B for Example 1D in Example 1E. MS (DCI/NH₃) m/z 333.0 (M+H)⁺; ¹H NMR (500 MHz, DMSO-d₆) δ 4.70 (d, J=3.12 Hz, 2H), 5.44 (d, J=4.68 Hz, 2H), 5.82 (m, 2H), 7.08 (dd, J=8.73, 2.81 Hz, 1H), 7.17 (d, J=8.73 Hz, 1H), 7.85 (s, 1H), 7.91 (s, 1H), 8.33 (d, J=2.50 Hz, 1H), 10.31 (s, 1H), 11.04 (s, 1H).

### EXAMPLE 5

### 7-chloro-11,16-dioxa-2,4,19,21-tetraazatricyclo[15.3.1.0^{5,10}]henicosa-1(21),5,7,9,17,19-hexaen-3-one

The desired product was prepared (10 mg, 50% yield) by substituting Example 4C for Example 1E in Example 2. MS (DCI/NH₃) m/z 335.0 (M+H)⁺; ¹H NMR (500 MHz, DMSO-d₆) δ 1.85 (m, 2H), 2.01 (m, 2H), 4.16 (t, J=7.2 Hz, 2H), 4.64 (t, J=5.6 Hz, 2H), 7.04 (dd, J=8.58, 2.65 Hz, 1H), 7.14 (d, J=8.73 Hz, 1H), 7.84 (s, 1H), 7.92 (s, 1H), 8.34 (d, J=2.50 Hz, 1H), 10.28 (s, 1H), 10.99 (s, 1H).

### EXAMPLE 6

### 7-chloro-13,14-dihydroxy-11,16-dioxa-2,4,19,21-tetraazatricyclo[15.3.1.0^{5,10}]henicosa-1(21),5,7,9,17,19-hexaen-3-one

The desired product was prepared (21.7 mg, 70% yield) by substituting Example 4C for Example 1E in Example 3. MS (DCI/NH₃) m/z 367.0 (M+H)⁺; ¹H NMR (400 MHz, DMSO-d₆) δ 4.06 (m, 3H), 4.18 (m, 2H), 5.14 (d, J=25.8 Hz, 1H), 5.19 (t, J=6.5 Hz, 1H), 5.28 (d, J=6.5 Hz, 1H), 7.06 (dd, J=8.75, 2.61 Hz, 1H), 7.20 (d, J=8.90 Hz, 1H), 7.87 (s, 1H), 7.92 (s, 1H), 8.36 (d, J=2.76 Hz, 1H), 10.28 (s, 1H), 10.88 (s, 1H).

### EXAMPLE 7

### 7-chloro-3-oxo-11,17-dioxa-2,4,20,22-tetraazatricyclo[16.3.1.0^{5,10}]docosa-1(22),5,7,9,13,18,20-heptaene-19-carbonitrile

### EXAMPLE 7A

### 2-chloro-3-(dichloromethyl)pyrazine

A mixture of 2-chloro-3-methylpyrazine (10 g, 77.8 mmol), acetic acid (90 mL), and chlorine (23.6 g) were combined and heated at 100° C. for 3 hours, and concentrated. The residue was suspended in dichloromethane, washed with water and 5% NaHCO₃, dried (MgSO₄), filtered, and concentrated. The crude product was purified by flash column chromatography eluting with hexanes/ethyl acetate (3:1) to provide the desired product (3.8 g, 25%). MS (DCI/NH₃) m/z 197.0 (M+H)⁺; ¹H NMR (400 MHz, benzene-d₆) δ 7.70 (s, 1H), 8.67 (d, J=2.15 Hz, 1H), 8.83 (d, J=2.45 Hz, 1H).

### EXAMPLE 7B

### 5-amino-3-chloro-2-pyrazinecarbaldehyde oxime

A solution of NH₂OH.HCl (7.04 g, 101.3 mmol) in H₂O (20 mL) and ethanol (20 mL) was buffered to pH 7.5 with 10M NaOH, treated with Example 7A (2 g, 10.13 mmol), heated to reflux at 95° C. for 6 hours, partially concentrated, and cooled to 0° C. overnight. The precipitate was collected by filtration and dried to provide the desired product (500 mg, 29%). MS (DCI/NH₃) m/z 173.0 (M+H)⁺; ¹H NMR (500 MHz, acetone-d₆) δ 7.26 (s, 2H), 7.88 (s, 1H), 8.15 (s, 1H), 11.40 (s, 1H).

### EXAMPLE 7C

### 5-amino-3-chloro-2-pyrazinecarbaldehyde O-acetyloxime

A solution of Example 7B (450 mg, 2.60 mmol) in 1N NaOH (10 mL) was treated dropwise with acetic anhydride (1 mL) while maintaining the temperature of the reaction mixture at 20° C. The reaction mixture was stirred for 10 minutes and the precipitate was collected by filtration, washed with water, and dried to provide the desired product (460 mg, 82%). MS (DCI/NH₃) m/z 215.12.0 (M+H)⁺; ¹H NMR (400 MHz, DMSO-d₆) δ 2.19 (s, 3H), 7.69 (s, 2H), 7.96 (s, 1H), 8.57 (s, 1H).

### EXAMPLE 7D

### 5-amino-3-chloro-2-pyrazinecarbonitrile

A suspension of Example 7C (460 mg, 2.14 mmol) in o-xylene (22 mL) was stirred at 160° C. for 20 hours, treated with charcoal (10 mg), and filtered. The filtrate was cooled to room temperature and the resulting precipitate was collected by filtration to provide the desired product (250 mg, 75%). MS (ESI) m/z 152.93 (M−H)⁻; ¹H NMR (500 MHz, DMSO-d6) δ 7.88 (s, 1H), 8.09 (s, 2H).

### EXAMPLE 7E

### 5-amino-3-(3-butenyloxy)-2-pyrazinecarbonitrile

A suspension of NaH (60%, 52 mg, 1.3 mmol) in dioxane (3 mL) in a microwave vial was treated with 3-buten-1-ol (0.112 mL, 1.3 mmol), stirred at room temperature for 30 minutes, and treated with Example 7D (100 mg, 0.65 mmol). The resulting mixture was heated to 100° C. for 3000 seconds in a Smith Synthesizer, cooled, and concentrated. The residue was purified by flash column chromatography eluting with hexanes/ethyl acetate (1:1) to provide the desired product (74 mg, 60%). MS (DCI/NH₃) m/z 208.12 (M+NH₄)⁺; ¹H NMR (500 MHz, acetone-d₆) δ 2.49 (m, 2H), 4.35 (t, J=6.71 Hz, 2H), 5.09 (dd, J=10.22, 1.98 Hz, 1H), 5.15 (dd, J=17.09, 1.83 Hz, 1H), 5.85 (m, 1H), 7.51 (s, 1H), 7.66 (s, 2H).

### EXAMPLE 7F

### phenyl 6-(3-butenyloxy)-5-cyano-2-pyrazinylcarbamate

A suspension of Example 7E (200 mg, 1.05 mmol) in pyridine (0.17 mL, 2.1 mmol) and CH₂Cl₂ (10 mL) at 0° C. was treated dropwise with phenyl chloroformate (0.145 mL, 2.1 mmol), stirred at room temperature for 3 hours, and directly applied to a flash column. The column was eluted with dichloromethane to provide the desired product (130 mg, 40%). MS (DCI/NH₃) m/z 328.13 (M+NH₄)⁺; ¹H NMR (500 MHz, DMSO-d₆) δ 2.56 (q, J=6.71 Hz, 2H), 4.49 (t, J=6.71 Hz, 2H), 5.11 (dd, J=10.22, 1.68 Hz, 1H), 5.19 (dd, J=17.24, 1.68 Hz, 1H), 5.88 (m, 1H), 7.27 (d, J=7.63 Hz, 2H), 7.31 (t, J=7.32 Hz, 1H), 7.47 (t, J=7.93 Hz, 2H), 8.73 (s, 1H), 11.62 (s, 1N).

### EXAMPLE 7G

### N-[2-(allyloxy)-5-chlorophenyl]-N′-[6-(3-butenyloxy)-5-cyano-2-pyrazinyl]urea

A mixture of Example 1A (108.8 mg, 0.59 mmol) and Example 7F (116 mg, 0.37 mmol) in toluene (10 mL) was heated to 90° C. for 24 hours and concentrated. The residue was purified by flash column chromatograghy eluting with hexanes/ethyl acetate (3:1) to provide the desired product (86 mg, 58%). MS (DCI/NH₃) m/z 400.09 (M+H)⁺; ¹H NMR (400 MHz, DMSO-d₆) δ 2.53 (q, J=6.65 Hz, 2H), 4.45 (t, J=6.60 Hz, 2H), 4.70 (m, 2H), 5.10 (dd, J=10.28, 1.99 Hz, 1H), 5.17 (m, 1H), 5.30 (m, 1H), 5.42 (m, 1H), 5.85 (m, 1H), 6.07 (m, 1H), 7.06 (d, J=2.15 Hz, IH), 7.06 (s, IH), 8.19 (d, J=2.15 Hz, 1H), 8.86 (s, 1H), 9.05 (s, 1H), 10.69 (s, 1H).

### EXAMPLE 7H

### 7-chloro-3-oxo-11,17-dioxa-2,4,20,22-tetraazatricyclo[16.3.1.0^{5,10}]docosa-1(22),5,7,9,13,18,20-heptaene-19-carbonitrile

The desired product was prepared (40 mg, 66% yield) by substituting Example 7G for Example 1D in Example 1E. MS (DCI/NH₃) m/z 389.09 (M+NH₄)⁺; ¹H NMR (500 MHz, DMSO-d₆) δ 2.71 (q, J=7.49 Hz, 2H), 4.68 (t, J=7.05 Hz, 2H), 4.70 (d, J=6.86 Hz, 2H), 6.02 (m, 1H), 6.09 (m, 1H), 7.12 (dd, J=8.89, 2.65 Hz, 1H), 7.22 (d, J=9.04 Hz, 1H), 7.98 (s, 1H), 8.12 (d, J=2.49 Hz, 1H), 10.35 (s, 1H), 10.97 (s, 1H).

### EXAMPLE 8

### 7-chloro-3-oxo-11,17-dioxa-2,4,20,22-tetraazatricyclo[16.3.1.0^{5,10}]docosa-1(22),5,7,9,18,20-hexaene-19-carbonitrile

The desired product was prepared (7 mg, 70% yield) by substituting Example 7H for Example 1E in Example 2. MS (DCI/NH₃) m/z 391.3 (M+NH₄)⁺; ¹H NMR (500 MHz, DMSO-d₆) δ 1.61 (dd, J=12.16, 6.24 Hz, 2H), 1.82 (m, 2H), 1.95 (m, 2H), 4.19 (t, J=5.15 Hz, 2H), 4.62 (t, J=8.45 Hz, 2H), 7.13 (m, 2H), 7.99 (s, 1H), 8.20 (d, J=2.18 Hz, 1H), 9.95 (s, 1H), 10.94 (s, 1H).

### EXAMPLE 9

### 7-chloro-13,14-dihydroxy-3-oxo-11,17-dioxa-2,4,20,22-tetraazatricyclo[16.3.1.0^{5,10}]docosa-1(22),5,7,9,18,20-hexaene-19-carbonitrile

The desired product was prepared (8.5 mg, 88% yield) by substituting Example 7H for Example 1E in Example 3. MS (ESI) m/z 404.01 (M−H)⁻; ¹H NMR (500 MHz, DMSO-d6) δ 1.93 (m, 1H), 2.33 (m, 1H), 3.82 (m, 2H), 4.12 (m, 2H), 4.61 (m, 1H), 4.69 (m, 1H), 4.90 (d, J=4.99 Hz, 1H), 5.08 (d, J=4.99 Hz, 1H), 7.14 (dd, J=8.89, 2.34 Hz, 1H), 7.18 (d, J=9.05 Hz, 1H), 8.00 (s, 1H), 8.16 (d, J=2.49 Hz, 1H), 9.79 (s, 1H), 10.93 (s, 1H).

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A compound of formula (I) 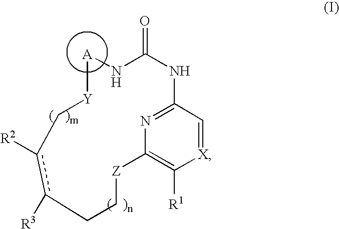
or a therapeutically acceptable salt thereof, wherein 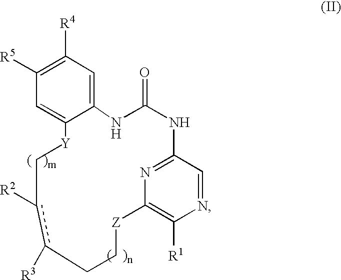
is a single or double bond;
A is selected from the group consisting of aryl and heteroaryl, wherein the aryl and the heteroaryl are optionally substituted with one, two, or three substituents independently selected from the group consisting of alkoxy, alkyl, cyano, halo, hydroxy, and NR^{a}R^{b};
R¹ is selected from the group consisting of hydrogen, alkoxycarbonyl, alkyl, carboxy, cyano, halo, and nitro;
R² and R³ are independently selected from the group consisting of hydrogen, alkoxy, alkoxycarbonyl, alkyl, alkylsulfonyl, arylsulfonyl, halo, hydroxy, and NR^{a}R^{b};
X is selected from the group consisting of CH and N;
Y and Z are independently selected from the group consisting of CH₂, O, and NR^{z}, wherein R^{z} is selected from the group consisting of hydrogen and alkyl; and
the sum of m and n is between 0 and 6, inclusive.

2. A compound of formula (II) 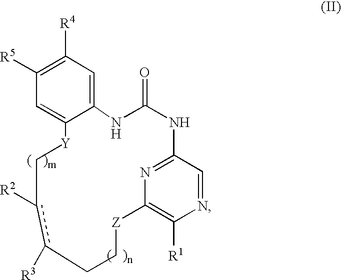
or a therapeutically acceptable salt thereof, wherein 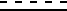
is a single or double bond;
R¹ is selected from the group consisting of hydrogen, alkoxycarbonyl, alkyl, carboxy, cyano, halo, and nitro;
R² and R³ are independently selected from the group consisting of hydrogen, alkoxy, alkoxycarbonyl, alkyl, alkylsulfonyl, arylsulfonyl, halo, hydroxy, and NR^{a}R^{b};
R⁴ is selected from the group consisting of alkoxy, alkyl, cyano, halo, hydroxy, and NR^{a}R^{b};
R⁵ is selected from the group consisting of hydrogen, alkoxy, alkoxyalkyl, alkoxycarbonyl, alkoxycarbonylalkyl, alkylsulfanyl, arylsulfanyl, alkylsulfonyl, arylsulfonyl, carboxy, carboxyalkyl, hydroxy, hydroxyalkyl, NR^{a}R^{b}, (NR^{a}R^{b})alkoxy, (NR^{a}R^{b})alkyl, (NR^{a}R^{b})carbonyl, (NR^{a}R^{b})carbonylalkoxy, and (NR^{a}R^{b})carbonylalkyl;
Y and Z are independently selected from the group consisting of CH₂, O, and NR^{z}, wherein R^{z} is selected from the group consisting of hydrogen and alkyl; and
the sum of m and n is between 0 and 6, inclusive.

3. The compound of claim 2 wherein
R¹ is selected from the group consisting of hydrogen and cyano;
R² and R³ are independently selected from the group consisting of hydrogen and hydroxy;
R⁴ is halo;
R⁵ is hydrogen;
Y is O;
Z is O; and
m is 1.

4. The compound of claim 1 wherein m is 1 and n is 0.

5. The compound of claim 4 selected from the group consisting of
7-chloro-11,16-dioxa-2,4,19,21-tetraazatricyclo[15.3.1.0^{5,10}]henicosa-1(21),5,7,9,13,17,19-heptaen-3-one;
7-chloro-11,16-dioxa-2,4,19,21-tetraazatricyclo[15.3.1.0^{5,10}]henicosa-1(21),5,7,9,17,19-5 hexaen-3-one; and
7-chloro-13,14-dihydroxy-11,16-dioxa-2,4,19,21-tetraazatricyclo[15.3.1.0^{5,10}]henicosa-1(21),5,7,9,17,19-hexaen-3-one.

6. The compound of claim 1 wherein m is 1 and n is 1.

7. The compound of claim 6 selected from the group consisting of
7-chloro-11,17-dioxa-2,4,20,22-tetraazatricyclo[16.3.1.0^{5,10}]docosa-1(22),5,7,9,13,18,20-heptaen-3-one;
7-chloro-11,17-dioxa-2,4,20,22-tetraazatricyclo[16.3.1.0^{5,10}]docosa-1(22),5,7,9,18,20-5 hexaen-3-one;
7-chloro-13,14-dihydroxy-11,17-dioxa-2,4,20,22-tetraazatricyclo[16.3.1.0^{5,10}]docosa-1(22),5,7,9,18,20-hexaen-3-one;
7-chloro-3-oxo-11,17-dioxa-2,4,20,22-tetraazatricyclo[16.3.1.0^{5,10}]docosa-1(22),5,7,9,13,18,20-heptaene-19-carbonitrile;
7-chloro-3-oxo-11,17-dioxa-2,4,20,22-tetraazatricyclo[16.3.1.0^{5,10}]docosa-1(22),5,7,9,18,20-hexaene-19-carbonitrile; and
7-chloro-13,14-dihydroxy-3-oxo-11,17-dioxa-2,4,20,22-tetraazatricyclo[16.3.1.0^{5,10}]docosa-1(22),5,7,9,18,20-hexaene-19-carbonitrile.

8. A pharmaceutical composition comprising a compound of claim 1 or a therapeutically acceptable salt thereof, in combination with a therapeutically acceptable carrier.

9. A method for inhibiting protein kinases in a patient in recognized need of such treatment comprising administering to the patient a therapeutically acceptable amount of a compound of claim 1, or a therapeutically acceptable salt thereof.

10. A method for treating cancer in a patient in recognized need of such treatment comprising administering to the patient a therapeutically acceptable amount of a compound of claim 1, or a therapeutically acceptable salt thereof.
